(19) United States
(12) Patent Application Publication
(10) Pub. No.: US 2015/0305700 A1
(43) Pub. Date: Oct. 29, 2015

(54) HYBRID IMAGING SYSTEM AND METHOD FOR INTRAOPERATIVE, INTERVENTIONAL, AND DIAGNOSTIC APPLICATIONS

(71) Applicant: SURGICEYE GMBH, München (DE)

(72) Inventors: Thomas WENDLER, München (DE); Stefan WIESNER, Markt Schwaben (DE); Jörg TRAUB, München (DE); Martin FREESMEYER, Jena (DE)

(21) Appl. No.: 14/647,070

(22) PCT Filed: Nov. 25, 2013
(86) PCT No.: PCT/EP2013/074626

§ 371 Date: May 22, 2015

(30) Foreign Application Priority Data
Nov. 23, 2012 (DE) 10 2012 111 386.6

(51) Int. Cl. A61B 6/00; A61B 8/08; A61B 8/00; G06T 11/00; A61B 6/03

(52) U.S. Cl. CPC A61B 6/4417; G06T 11/003; A61B 6/037; A61B 6/4405; A61B 6/547; A61B 8/4254; A61B 8/4263; A61B 8/0841; A61B 8/5261; G06T 2207/10081

(57) ABSTRACT

A combined nuclear and ultrasonic system for hybrid imaging is proposed. It comprises a hand-held nuclear radiation detector, a hand-held ultrasonic probe, a nuclear tracking system for tracking the nuclear radiation detector while measuring a nuclear radiation and obtaining nuclear radiation detector coordinates representing a position of the tracked nuclear radiation detector in relation to an image coordinate system of a hybrid image, an ultrasonic tracking system for tracking of the ultrasonic probe during the receiving of ultrasonic signals so that ultrasonic probe coordinates are obtained which represent a position of the tracked ultrasonic probe in relation to an image coordinate system of the hybrid image, and a data acquisition module, which gathers the nuclear radiation detector measurements, nuclear radiation detector coordinates, ultrasonic signals from the ultrasonic probe, and ultrasonic probe coordinates, and matches them with an image coordinate system of a hybrid image. A nuclear image reconstruction module is for reconstructing a nuclear image from nuclear radiation detector measurements, ultrasonic signals, nuclear radiation detector coordinates and ultrasonic probe coordinates in the image coordinate system of the hybrid image, and an evaluation system calculates a nuclear-image-quality-value of nuclear radiation detector measurements and nuclear radiation detector coordinates. Further, a method for hybrid imaging is proposed.

This disclosure relates to the field of imaging, especially to the field of intraoperative, interventional and diagnostic imaging with hand-held detectors.

In diagnostic imaging, combinations of different methods are increasingly being used, the so-called hybrid imaging systems. Above all, systems are to be combined which are complementary to one another, and in which the disadvantages of one system are compensated by the advantages of the other, or the advantages of both systems can be combined, so that the new system is better than the single application.

For example, with X-ray computed tomography (CT), morphological structures can be represented with a high spatial resolution, with the disadvantage of relatively poor soft tissue contrast, lack of functional information, the use of nephrotoxic contrast agents and the presence of harmful gamma radiation (radiation exposure).

Although a nuclear medicine system also uses harmful radiation, it can represent functional information much better, however with a rather poor spatial resolution. A combination of a CT scanner and a nuclear medicine system (such as a single-photon emission computed tomography system, SPECT, or a positron emission tomography system, PET) could thus at least provide a high resolution image with functional information. There is of course the possibility to acquire the respective image data separately and to combine subsequently (image registration). However, this may have the disadvantage of poor correlation due to an intermediate deformation or movement of the patient body, and due to a differing positioning of the patient. Thus, the accuracy of such image data is very limited. Ideally, the imaging is performed by a combined system, such as is available through the so called PET/CT or SPECT/CT systems since several years. Thereby, a nuclear image and a CT scan are performed sequentially in a combined system. Under sequential recording, one refers to a recording where the recordings of both imaging methods do not overlap in time, but one after the other is carried out with a few minutes difference.

The combination of magnetic resonance imaging (MRI) and PET in an integrated system (MR/PET system) has shown that the disadvantages of CT (limited soft tissue contrast, nephrotoxic contrast agents, gamma radiation) can be prevented. The MR system can, on the one hand, produce very high spatial resolution images with an excellent soft tissue contrast. In addition, the application of magnetic resonance imaging by the wavelength and energy of the quantums employed is harmless. With nuclear medicine, this can now be provided with additional functional information of a specific area. Specifically in oncology, this not only helps with the initial diagnosis, but also in the follow-up control of a subsequent therapy.

Another advantage of MR/PET systems is that one can perform the data acquisition of MR and PET jointly in certain implementations. This prevents a poor correlation with intervening deformation or movement of the patient, and by a varying positioning of the patient. Under a joint recording, a recording is understood in which the recording of at least two imaging modalities overlaps during a time interval. For example, in an MR/PET, the PET images can be acquired before or after the MR recording, or even simultaneously with the MR recording. Similarly, the MR recording can be carried out prior to or after the PET recording, or simultaneously with it.

The problem of currently used PET/CT, SPECT/CT, or MR/PET systems is their exclusive use for diagnostic tasks, since they cannot be used intraoperatively or interventionally in a flexible manner.

The same is true for systems such as described in US Patent Application 2010/0016765 A1, or in U.S. Pat. No. 6,455,856 where an ultrasonic system is installed in a stationary SPECT system or in the stationary gamma camera.

In a diagnosis carried out and a subsequent interventional procedure or surgery of a tumor, although the image data would be available before the intervention/operation, changes that could occur after imaging and before the intervention/operation cannot be shown. Such changes may be effected by normal shifts of organs, varying positioning (e.g. Intervention/operating table is just, bed of the diagnostic system includes various positioning aids, diagnosis is carried out with the arms above the head, but the therapy with the arms at the side, or vice versa).

In addition, it would be desirable to have, even during the intervention/operation, information about the success of the operation (tumor tissue visually differs not necessarily of normal tissue), and to ensure that all diseased tissue has been removed but then to leave as much healthy tissue in the body. This can actually only be achieved in that the imaging is also available during the intervention/operation. In addition, one can see the picture information, of course, to take advantage of the intervention/operation volume being clearly labeled and to navigate the intervention/operation tool correspondingly exactly to this volume.

Many other therapeutic methods, among others, in neurology and orthopedics have similar problems, and could benefit from such a combined system in the direct operation environment.

The lack of mobility of the equipment is in itself a disadvantage in diagnostics. It is often desired in elderly or very ill patients, that image recordings (or even intervention) is performed at the bedside. Also, in smaller hospitals, equipment is often shared, and it is thus advantageous to be able to move these.

Another problem of these hybrid systems is the high cost of the machines. This allows their use only in large institutions or specialized centers. Thus, only a subgroup of all patients can be diagnosed.

As an alternative to this type of expensive permanently installed/stationary hybrid imaging systems, ultrasound systems have been upgraded to load and display PET/CT or SPECT/CT data in recent times. Current ultrasound systems are often upgraded with positioning systems in order to be able to display in real-time the location and orientation of the ultrasound probe. Via registration methods such as point-based registration or intensity-based registration, the CT data is associated with the ultrasound images and thus, over the known registration of PET or SPECT with CT and the calculated registration from CT to ultrasound, equivalent PET/Ultrasound and SPECT/ultrasound images may be generated. Examples of such systems are the GE Logiq E9 system, or the system of the European patent application EP 2104919 A2.

However, this strategy still bears problems: First, it needs a CT, which means a radiation exposure to the patient just for merging the ultrasound images with the nuclear medicine images. Secondly, the functional images (PET or SPECT) are often taken a few days before the ultrasound images, so that a fusion of the data is only partially valid. Thirdly, with the step of registering, these systems necessarily incorporate correlation errors that render a 100% fusion of the data impossible.

Relevant background to this invention is the freehand SPECT technology (T. Wendler, A. Hartl, T. Lasser, J. Traub, F. Daghighian, S. I. Ziegler, N. Navab; Towards intraoperative 3D nuclear imaging: reconstruction of 3D radioactive distributions using tracked gamma probes; Proceedings of Medical Image Computing and Computer-Assisted Intervention (MICCAI 2007), Brisbane, Australia, Oct. 29-Nov. 2, 2007, LCNS 4792 (2), pp. 252-260//T. Wendler, K. Herrmann, A. Schnelzer, T. Lasser, J. Traub, 0. Kutter, A. Ehlerding, K. Scheidhauer, T. Schuster, M. Kiechle, M. Schwaiger, N. Navab, S. I. Ziegler, A. K. Buck; First demonstration of 3-D lymphatic mapping in breast cancer using freehand SPECT; European Journal of Nuclear Medicine and Molecular Imaging, Springer Berlin/Heidelberg, 2010 August; 37(8):1452-61). It allows to generate SPECT equivalent images, but is based on hand-held detectors, such as a gamma probe rather than large gamma cameras that are used in conventional SPECT. These detectors can be located by a positioning system, so that their measurements are complemented by their position and orientation. From the measurements of the detectors, their respective positions and orientations, SPECT images are generated. The concept of freehand SPECT can also benefit from the use of coincidence detectors, wherein at least one of those is hand-held, and be extended to a freehand PET imaging. Freehand SPECT and PET systems are less expensive than their conventional stationary alternatives.

In 2009, a freehand SPECT system has been upgraded for additionally tracking an ultrasound probe (T. Wendler, T. Lasser, J. Traub, S. I. Ziegler, N. Navab; Freehand SPECT/ultrasound fusion for hybrid image-guided resection; Proceedings of Annual Congress of the European Association of of Nuclear Medicine—EANM 2009, Barcelona, Spain, October 2009). By calibration, it was possible to demonstrate the plausibility of a freehand SPECT/ultrasound imaging.

This prototype had the problem of sequentiality of data collection: first a freehand SPECT image was generated, and only then the ultrasound images were taken. The image quality was not particularly good, since deformation occurred and the patient moved between the two shots. Furthermore, the demands on the image quality of the freehand SPECT were high, so that an image reconstruction only based on the gamma-probe measurements and the position and orientation of the gamma probe was able to resolve resolutions of >7 mm, and could only make great contrasts visible.

Similar ideas were embodied in U.S. Pat. No. 6,512,943. Therein, an ultrasound system was mechanically coupled with two nuclear radiation detectors. From measurements of the nuclear radiation detectors, the depth of a radioactive source can be determined. With the ultrasound image that delivers the ultrasound system, one can then make an allocation which structures are radioactive, and even carry out biopsies. The main problem of such a system is that it can only be used in locating individual radioactive point sources, since it covers the 3D position of only two values.

In the U.S. Pat. No. 4,995,396, an endoscope integrates an optical camera, an ultrasound device, and a gamma camera. The system should be able to also represent the images of the gamma camera along with the images of the endoscope and ultrasound. This is in practice not possible, because the ultrasound images are sectional views in depth, and the gamma camera images are projected images.

In the U.S. Pat. No. 6,628,984, a hand-held gamma camera is described, which is tracked in order to reconstruct tomographic images. These images can be understood as 3D nuclear images such as freehand SPECT or PET Freehand, and can also be registered with ultrasound images, according to the inventors. This approach is basically the same that was implemented in the commercial ultrasound systems, which were discussed above. It captures the data sequentially, and suffers from problems with movements and deformations of the patient. Since this system is similar to the above-mentioned freehand SPECT system of the group of T. Wendler et al., it is also expected to provide only a low resolution and poor contrast in a real implementation.

In view of the above, a combined nuclear and ultrasound system for hybrid imaging according to claim 1, and a method for hybrid imaging according to claim 10 is proposed.

### BRIEF DESCRIPTION OF DRAWINGS

In the following, the invention will be described with reference to exemplary embodiments illustrated in figures, from which further advantages and modifications will readily occur.

FIG. 1 shows the connections of different components according to an embodiment of the invention.

FIG. 2 illustrates an embodiment of the invention, where the nuclear tracking system (40) and the ultrasonic tracking system (50) are a single tracking system.

FIG. 3 illustrates an embodiment of the invention, which is similar to that of FIG. 2, but a reference to an object or living being (81).

FIG. 4 shows a fixing device (82) in accordance with embodiments to stabilize the object or the living being (80) during the measurement of the nuclear radiation.

FIG. 5 shows a further fixation device (82) according to embodiments, to stabilize the object or the living being (80) during the measurement of the nuclear radiation or the reference for an object includes living beings (81).

FIG. 6 shows a manner in accordance with embodiments to produce a hybrid image.

FIG. 7 shows a possible sequence of steps of data acquisition in accordance with embodiments of the invention.

FIG. 8 shows another possible sequence of steps of data acquisition in accordance with embodiments of the invention.

FIG. 9 presents a combined probe according to embodiments where nuclear radiation detector and ultrasonic probe are integrated in a hand-held probe.

FIG. 10 presents another combined probe of embodiments where nuclear radiation detector and ultrasonic probe are integrated in a hand-held probe.

FIG. 11 shows how, according to embodiments, segmentation can be calculated for nuclear image reconstruction of a power Doppler ultrasonics image.

FIG. 12 shows how, according to embodiments, segmentation can be calculated for nuclear image reconstruction of a B-mode image of ultrasonics.

FIG. 13 illustrates an embodiment of the invention, where all components are carried out separately.

FIG. 14 shows a further embodiment of the invention, where the nuclear tracking system (40) and the ultrasonic tracking system (50) are a single tracking system.

### REFERENCE NUMERALS IN THE FIGURES

(10) combined nuclear and ultrasound system
(11) hybrid image
(12) overlap area of the hybrid image, where nuclear-image and ultrasonic signals
(20) hand-held nuclear radiation detector
(21) Nuclear Detector measurements
(24) detector material from nuclear radiation detector
(25) Electronics of the nuclear radiation detector
(26) shield of the nuclear radiation detector
(27) collimator of the nuclear radiation detector
(30) hand-held ultrasound probe
(31) ultrasonic signals
(32) ultrasonic emitter/detector
(35) electronics of the ultrasound probe
(40) Nuclear tracking system
(41) Nuclear-detector coordinates
(42) Stationary part of the nuclear tracking system
(43) Mobile part of the nuclear tracking system on nuclear detector
(44) Mobile part of the nuclear-tracking system on reference from living beings or object
(50) ultrasonic tracking system
(51) Ultrasonic probe coordinates
(52) Stationary part of the ultrasonic tracking system
(53) Mobile part of the ultrasonic tracking system on ultrasonic probe
(54) Mobile part of the ultrasonic tracking system on reference from living beings
(60) Data Acquisition Module
(61) Complete data
(70) image reconstruction module
(71) 3D nuclear image
(72) attenuation map or stray card
(80) object or living being
(81) a reference of an object or living being
(82) fixation device
(90) output system
(100) surgical or interventional instrument

### DETAILED DESCRIPTION OF THE FIGURES

In the following, various embodiments of the invention are described, some of which are exemplified in the figures. In the following description of the figures, like reference numerals refer to like or similar components. In general, only differences between the various embodiments will be described. Here, features that are described as part of one embodiment, can also readily be combined in connection with other embodiments, in order to produce still other embodiments.

In embodiments, among other things, a hybrid system of a nuclear radiation detection system and an ultrasound system are proposed, which have the benefits of nuclear medicine (functional diagnostics mainly by tumor foci, possibility of flexible and individual handling on hand-held detectors, no magnetic fields and thus use of sensitive electronic peripheral systems) and ultrasonic systems (excellent soft tissue contrast, very high spatial resolution, flexibility, low cost) combined on a common system. Both imaging modalities can be incorporated into a common data acquisition, and are connected, and thus the possibility for compensation of movement and deformation is provided, particularly in sequential recordings.

In the proposed system and method according to embodiments is the combination of a freehand nuclear medicine system (such as freehand SPECT or freehand PET) and an ultrasound system that use a common reference system. For this, a nuclear radiation detector, and an ultrasonic probe are functionally connected, wherein the position and orientation of the nuclear radiation detector and the ultrasonic probe are detected by a respective positioning system each, in real time. From the measurements (detected radiation and ultrasonic signals) of both systems and the information of the position and orientation of the radiation detector and the ultrasonic probe with respect to a common reference, 3D tomographic images of the distribution of radiation can be generated in a living being or object. These images are also visualized together with the ultrasonic signals in the form of a hybrid image.

A combination of nuclear detection and ultrasonic detection as outlined above allows functional data (such as freehand SPECT or freehand PET images) and anatomical images (such as generated from the ultrasonic signals) to acquire, reconstruct and visualize together in real time or quasi-real time. With such a system, one can perform the data acquisition and visualization in a single step, i.e. the images of different modalities can be recorded jointly within a few seconds of one another, or even simultaneously, when one ultrasonic probe and nuclear radiation detector can be used simultaneously or when both are incorporated in a probe. Furthermore, it is expected that the cost of implementing such a system are significantly lower than the cost of a PET/CT, SPECT/CT, or MR/PET.

Embodiments of the present invention are useful, by the included 3D image reconstruction (in the sense of freehand SPECT or freehand PET) to solve the problem, which is to display images of a gamma camera together or combined with the images of the endoscope and ultrasonic probe. In embodiments, the nuclear-image is a 3D image, which makes it possible to merge the ultrasonic sectional image and the otherwise projective nuclear information into a fusion or overlay. Fixed or stationary 3D nuclear imaging devices (such as SPECT, PET or their combinations with CT and MR) are generally less suitable for use in embodiments, since they are not flexible for intraoperative or interventional use, and also do not allow for common data acquisition of ultrasound and nuclear detector measurements due to their construction (as gantry).

In the context of this publication, the term “image” is understood to include Information on the distribution of a nuclear source and information from an ultrasonic signal which are included as a function of position for at least one image segment of the image. That is, an assignment H (x, y, z), wherein H is assigned to an area in space for the coordinates x, y, z, H comprises at least two values, and thereby at least one radioactivity density and echogenicity (a value of an ultrasound image).

In particular, the following aspects have to be considered in the implementation of embodiments of the invention. On the one hand the quality of the images of freehand nuclear imaging is highly dependent on the user. It requires therefore generally an evaluation system which is preferably continuously calculates the quality of the nuclear-image and, based on deciding whether the current database to display sufficient in accordance with defined or desired quality. Alternatively, and recommended is the use of a (e.g. visual or auditory) system instruction for the user, so that the user can optimize the quality of the resulting nuclear-image based on the instruction. To this end, a desired position and orientation of the nuclear radiation detector (20) are used for calculating at least one nuclear-image-quality-value, preferably in a continuous manner. Furthermore, based thereon an instruction may be provided/issued to the user, or optionally to a robot that guides the detector, and preferably continuously—in order to achieve a movement of the nuclear radiation detector to the desired position(s) and orientation(s), which permit/obtain the optimization of the quality value.

On the other hand, a non-one-dimensional motion and/or deformation of the object or living being (80) during the measurement act potentially has very negative effect on the quality of the obtained nuclear image. The present invention implements in some embodiments one or more fixing devices (82), so that the object or living being (80) fixedly remains stable during the measurement. In this way, the quality of the nuclear-image can be ensured.

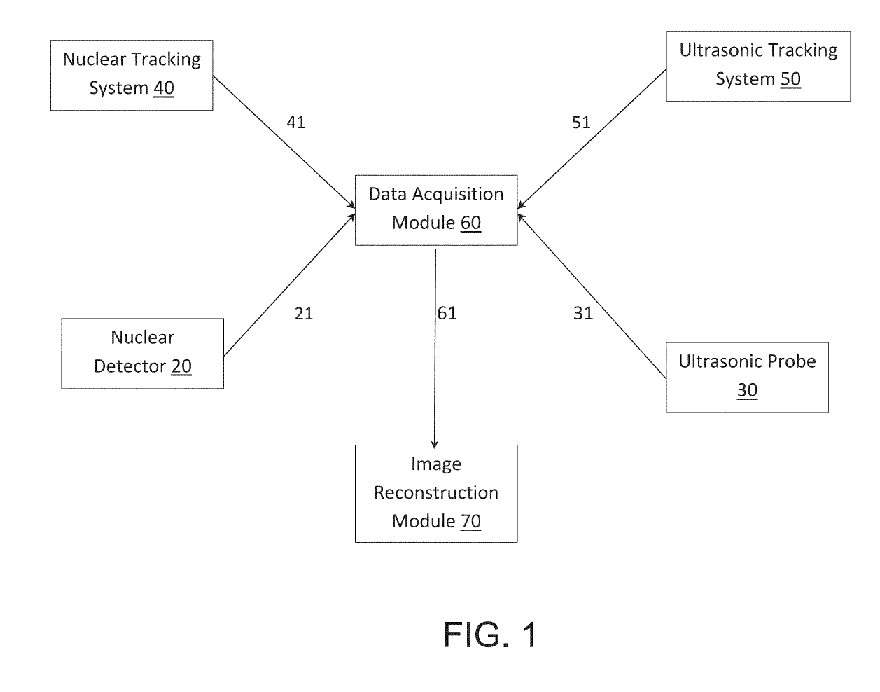

FIG. 1 shows the connections of the various components of embodiments of the invention. The nuclear detector (20) detects radiation in the form of nuclear detector measurements (21) which are sent to the data acquisition module (60). These detector measurements may be individual radiation values, such as is the case when the radiation detector is a gamma probe. In this case, the individual radiation-values are all gamma photons within an energy window in a time interval of one second, that is, the so-called count rate (Engl, “counts per second” or CPS). However, the detector measurements can also be 2D images, such as in the case that the nuclear radiation detector is a hand-held gamma camera. The gamma camera images would specify the count rate of each pixel of the camera.

The position and orientation of the nuclear detector (20), i.e. the nuclear detector coordinates (41) are detected by the nuclear-tracking system (40). These are usually a vector having a 3D position and 3 Euler angles. Alternatively, one can use a 4D quaternion, to describe the 3 Euler angles in a manner more numerically stable.

Both the detector measurements (21) and nuclear detector coordinates (41) are recorded by the data acquisition module (60). In an embodiment of the present invention the data acquisition module (60) can synchronize this data. Thus, each detector measurement can be assigned to a nuclear detector coordinate. A possible implementation of this is the use of tables, where all data will be stored and then mapping algorithms assign the closest nuclear detector coordinate to each detector measurement.

The nuclear detector measurements (21) and nuclear-detector coordinates (41) can be assigned to one another in a further embodiment of the invention by own timestamps of a common clock, of its own clocks with known timing differences, or after adoption of a known transmission delay. Alternatively, this data can be stored in a so-called ring buffer, with their own timestamps or new timestamps given by the data acquisition module (60), and are stored and assigned as needed. The “ring” in the name comes from the fact that old measurements are overwritten after a certain time period.

In a further embodiment of the invention, interpolation algorithms such as linear interpolation, or cubic interpolation algorithms, or algorithms using filters in the time domain, such as Kalman filter or particle filters, are employed for a better assignment of detector measurements (21) and nuclear detector coordinates (41).

On the side of the ultrasound, the ultrasound probe (30) delivers ultrasonic signals (31), such as, amongst others, linear measurements (A-mode ultrasound), 2D images (B-mode ultrasound), 2D Doppler images (normal Doppler, power Doppler, etc), Elastographic images or 3D images.

The position and orientation of the ultrasound probe (30) is also detected by a tracking system, namely, the ultrasonic tracking system (50).

The ultrasonic signals (31) and the ultrasonic probe coordinate (51) are also sent to the data acquisition module (60) and, in one embodiment of the invention, are synchronized with the nuclear detector measurements (21) and the nuclear detector coordinates (41).

In a further embodiment, the data acquisition module (60) preprocesses all the data from the data acquisition module (60), this data being designated together as a “full data” (61), such as by the use of filters to clear “Outliers” or eliminate various known noise signals from the complete data (61).

The complete data (61), whether preprocessed, synchronized or untouched, are then sent to the image reconstruction module (70). This module has in embodiments, multiple tasks:

1. It can determine a volume for image reconstruction. This may be predetermined, but can also be determined from the nuclear detector coordinates (41) and the ultrasonic probe coordinates (51), by accumulating which 3D positions were most often recorded by the nuclear detector (20) and/or the ultrasonic probe (30). For this, a calibration of the two hand-held parts is necessary in order to assign to where the field of vision of the two hand-held parts is directed, each relative to the elements tracked by the respective trackers. Other methods for image reconstruction can be found in the German application 102011053708.2 of one of the inventors of this invention.

2. It can use the information in the ultrasound signals (31) and the ultrasonic probe coordinates (51), e.g. for determining, in an embodiment, an attenuation map (or weakening card). A weakening card can be used later in the image reconstruction. The image reconstruction module (70) can in another embodiment calculate a scattering map from n ultrasonic signals (31) and the ultrasonic probes coordinates (51). Other tasks of the image reconstruction module (70) can be the segmentation of organs or parts thereof, the compensation of movements, etc.

3. It can be a nuclear image (71) from the nuclear detector measurements (21) and the nuclear detector coordinates (41) in consideration of the of the ultrasonic signals (31) and ultrasonic probe coordinates (51) information reconstruct (such as attenuation maps, scattering cards, segmentation of organs, estimated movements and/or deformations, etc.). To calculate the nuclear-image (71) the the nuclear image reconstruction module (70) can use conventional methods of image reconstruction, such as iterative image reconstruction methods. A pre-processing of the input data or a reworking of the reconstructed image data can also be implemented in the nuclear image reconstruction module (70). Examples of such preprocessing methods can be plausibility methods detecting non-plausible measurements, filtering techniques which smoothen potential noise in the recordings, information calculation methods that calculate the area where the calculating the nuclear-image has sufficient information, etc. Details on how such as a nuclear image can be calculated and how such filters can be applied, can be found in the German applications 102008025151 of a subgroup of the inventors of this invention. More details are also in the German application 102011053708.2 of one of the inventors of this invention.

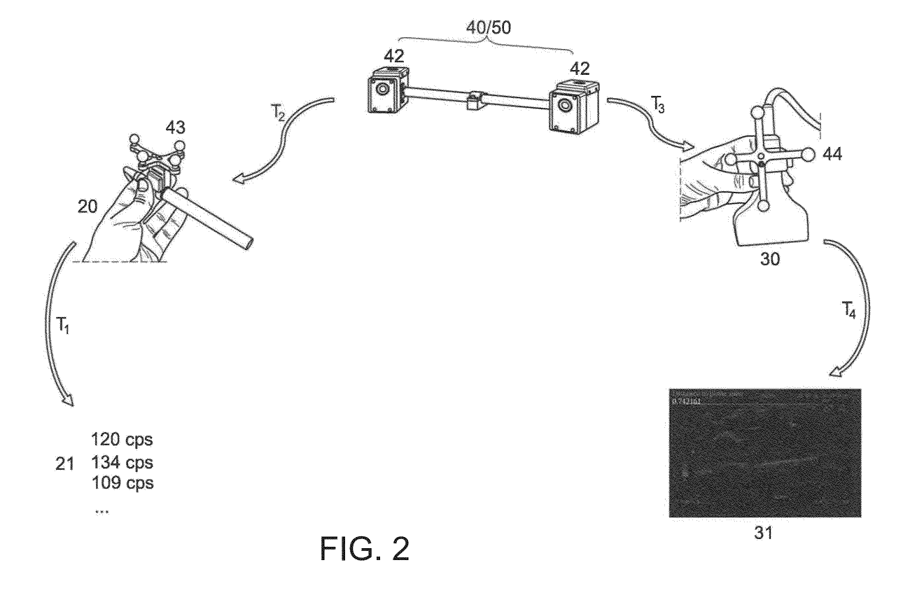

FIG. 2 shows embodiments of the invention, in which the nuclear tracking system (40) and the ultrasonic tracking system (50) are the same system.

Here, the nuclear radiation detector (20) is a conventional gamma-probe, the detected gamma radiation in the energy range 27-364 keV and has a lateral shielding, so that essentially only radiation from a narrow cone in the direction of the major axis of the gamma probe is measured. The nuclear measurements are counting rates in CPS.

The Nuclear Radiation Detector (20) is tracked by an optical passive localization system, here the design of the nuclear-tracking system (40). The nuclear tracking system (40) consists of a stationary tracking part (42), here e.g. two infrared reflectors on the nuclear detector (43) and on the ultrasonic probe (54).

This figure shows relatively clear how the different coordinates are converted to a common coordinate system, the coordinate system of the hybrid image. By calibration or mechanical drawings can be the transformation (eg., a 4×4 transformation matrix when using homogeneous coordinates) of the infrared reflectors in the nuclear detector (43) to the detector material determined by the nuclear radiation detector (24)—Transformation T1.

The transformation from the nuclear-tracking system (40) to the infrared reflectors on the nuclear detector (43)—Transformation T2—is determined in real time by the nuclear-tracking system (40).

Similar to the nuclear detector (20) from the nuclear tracking system (40), which is in this embodiment identical with the ultrasonic tracking system (50), tracked to the ultrasonic probe (30). For those with infrared reflectors (54) is upgraded. Thus, the transformation T3—are determined by the nuclear tracking system (40) to the infrared reflectors on the ultrasound probe (44).

By ultrasonic calibration methods such as a “Single-Wall-Calibration” or a grid-based calibration to transform the infrared reflectors on the ultrasound probe (54) to the plane of the ultrasound image (the ultrasonic signal (31) determine in this version)—transformation T4.

Thus, one can convert the nuclear detector measurements (21) and the ultrasonic signals (31) in any common coordinate system. There, they can then be used to reconstruct a hybrid image.

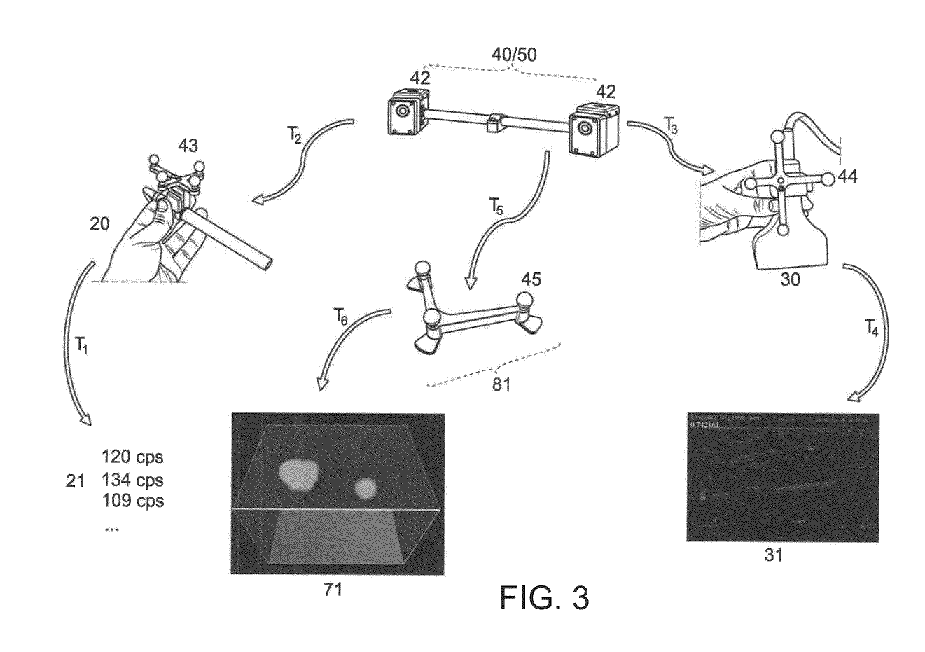

FIG. 3 shows an embodiment of the invention as shown in FIG. 2 with the difference that it is used a reference for the object or living being (81). This reference is tracked by the common tracking system, so that the transformation from the nuclear-tracking system (40) to the infrared reflectors on the reference to the object or living being (45)—Transformation T5—is determined by the tracking system.

By calibration or algorithms for calculating the volume where the hybrid image is reconstructed (such as in the description of FIG. 1) can also be determined T6. Thus, the complete data (61) can be converted into a common coordinate system.

The use of a reference (82) for the article or the animal brings advantages. For example, the object or living being (80) can move rigid without new data must be recorded. Furthermore, the tracking system also can move without losing the validity of the data collected up to that point.

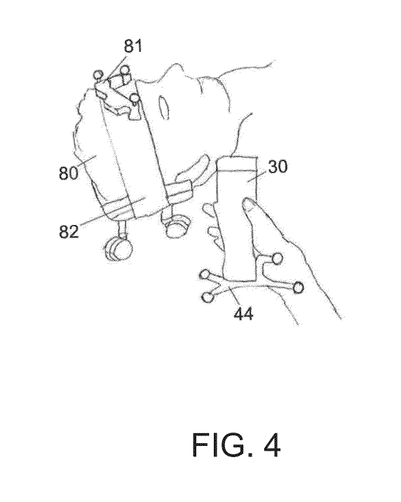

FIG. 4 shows a fixing device (82) to hold the object or living being (80) in embodiments stable stationary during the measurement of nuclear radiation. Non-rigid motions and deformations are not compensated by the use of the reference (81) for the object or the living being (80). For this reason, it makes sense to keep the object or living being (80) during the measurement of nuclear radiation stable stationary. Further, the fixing device S (82) also holds the object or the animal stable after measuring the nuclear radiation. This allows, among others to generate different sections of the hybrid image, e.g. for certain regions to be examined in more detail. A further advantage is that further nuclear data can be obtained, if a previous resolution of the nuclear image (71) must or shall be increased in certain regions.

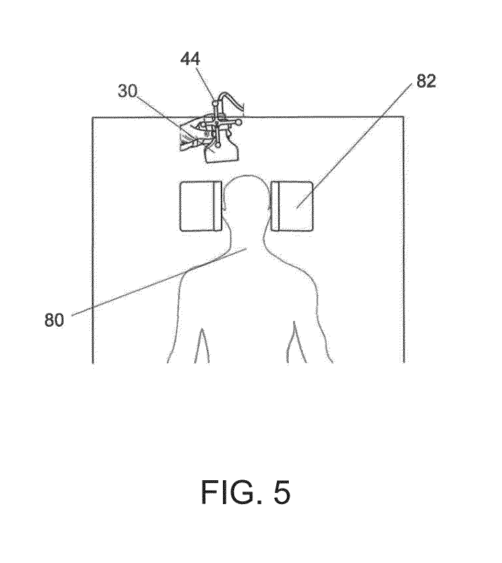

FIG. 5 shows a fixation device s (82), which also includes reference to an object or living being. This has the advantage that the reference to the object or living being not necessarily to the object or living being (80) must be attached (as in FIG. 4), and thus non-rigid motions and deformations of the surface of the object or living being (80), which could reduce the quality of the nuclear image (71), are minimized. Also for reasons of hygiene, this can be advantageous.

The fixation device (82) can be selected from the group consisting of:

A) mechanical mounts,

B) Vacuum pillows,

C) on the surface or a part of a tailor-made rigid mask formed from the object or living being (80),

D) Materials which are fixed under electric, magnetic or electromagnetic fields, but in their absence are deformable,

E). Materials which are in a temperature or pressure range determined, but are outside of those deformable

F) or a combination of any of the above fixation devices.

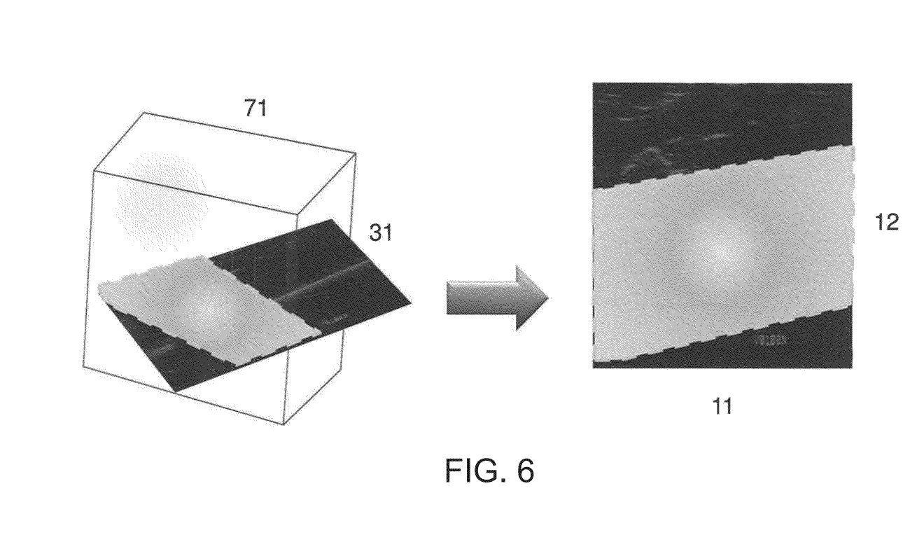

FIG. 6 shows a manner in accordance with embodiments of a reconstructed 3D nuclear image (71) and a 2D ultrasound image to generate a hybrid image. In this embodiment, the 3D-nuclear image (71) and an ultrasonic image (here the execution of the ultrasonic signals (31)) are converted to the same coordinates. The plane of the ultrasound image is then cut with the volume of the 3D-nuclear image. From the intersection of the nuclear-image and the ultrasonic signals (12) a 2D nuclear-image will be generated that can then be superimposed on the ultrasound image. The resulting image is thus a 2D ultrasound image (eg. in gray color) on which in the area of intersection of the nuclear-image and the ultrasonic signals (12) colored (shown in FIG. 6, for technical reasons in grayscale), the Radioactivity distribution of 3D-nuclear-image (71) are superimposed.

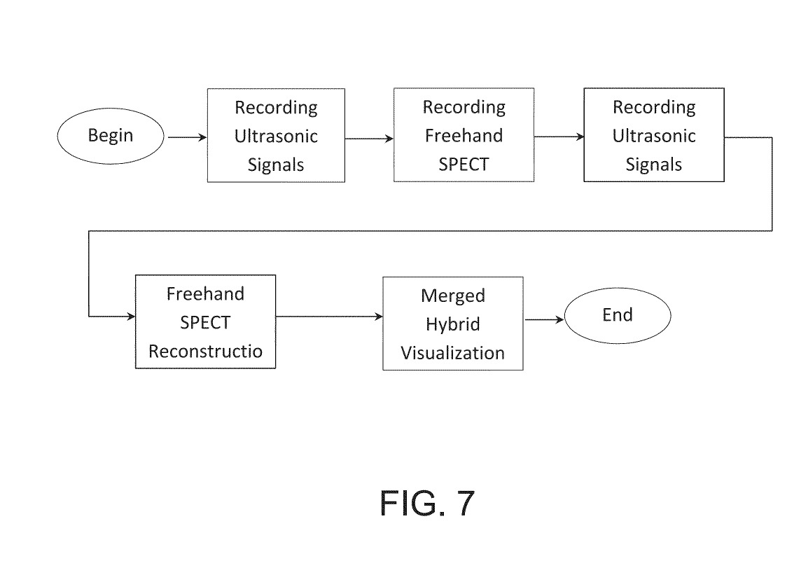

FIG. 7 shows a sequence of steps as embodiments of the invention can be used. A first step is made by moving the ultrasonic probe (30), and receiving ultrasonic. Then, without moving the animal or object (80) in this embodiment, the nuclear detector and nuclear detector measurements are employed. In this embodiment, the nuclear detector detects radiation in the energy range 27-364 keV, so the resulting nuclear imaging is freehand SPECT. Before the nuclear detector measurements are used in the proposed sequence of steps, a second ultrasound picture is taken. It serves to detect deformations and movements of the subject or the object (80) and can be adjusted accordingly, so the nuclear detector can compensate for the deformation and movements. At the end of the sequence of steps, a 3D-nuclear-image (71) is reconstructed with the information of the deformation and movement and then the ultrasonic signals and the 3D-nuclear-image are shown merged.

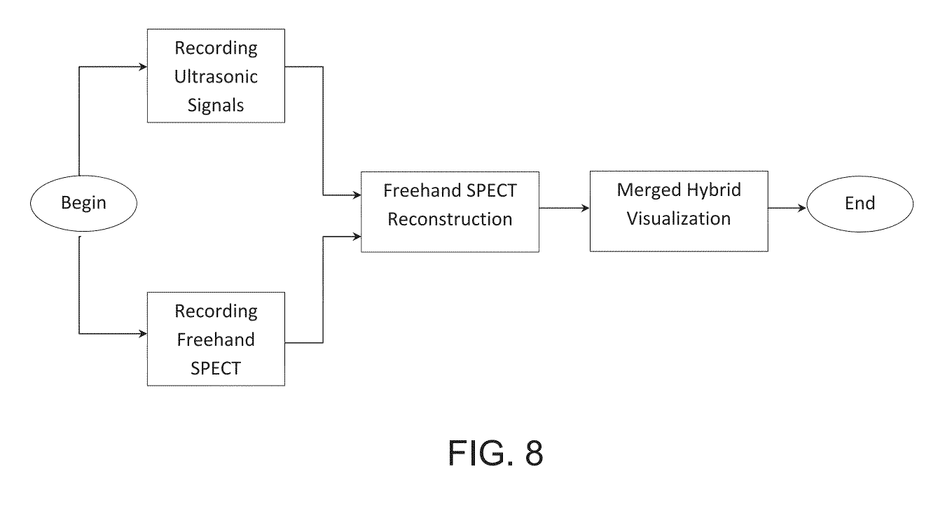

FIG. 8 shows another sequence of steps, which run parallel in contrast to the sequence of steps of FIG. 7, the ultrasonic recording and the freehand SPECT recording. This is possible when the nuclear detector (20) and ultrasonic probe (30) moves either simultaneously, or mechanically coupled. Two possible implementations of this mechanical coupling can be seen in FIGS. 7 and 8.

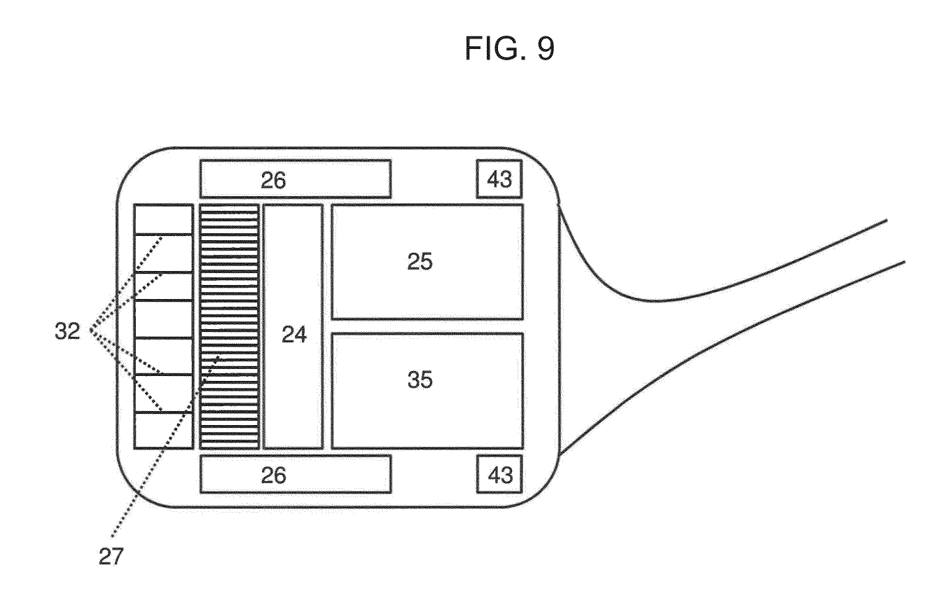

FIG. 9 shows a mechanically coupled nuclear-detector ultrasonic probe pair according to embodiments. In a housing are on one side ultrasonic emitter/detectors applied (32), such as piezoelectric crystals. These can (not in picture) generate ultrasound images associated with an ultrasonic electronics (35) and a computer unit. Behind the ultrasonic emitter/detectors (32), a collimator (27) installed which allows only nuclear radiation from a direction in nuclear radiation detector's detector material (24). The nuclear radiation to the detector material (24) is then detected and processed the resultant signal by the electronics from the nuclear radiation detector (26) in nuclear detector measurements (21).

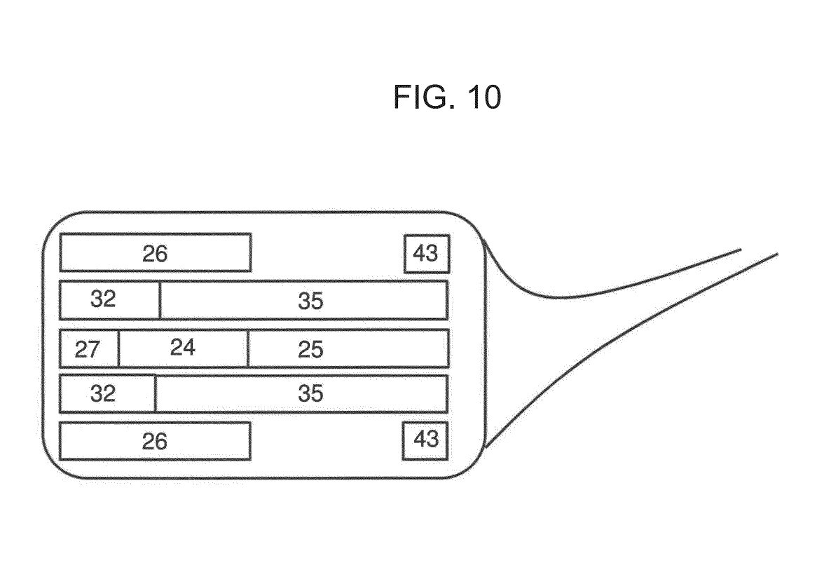

FIG. 10 shows a further embodiment of a nuclear detector mechanically coupled ultrasonic probe pair. Here the housing is much smaller than in the embodiment of FIG. 9. The nuclear detector (20) is even a “OD” detector. The ultrasonic probe (30) here consists of a ring of ultrasonic emitter/detectors to the collimator (27) and the material of the nuclear detector (24) placed.

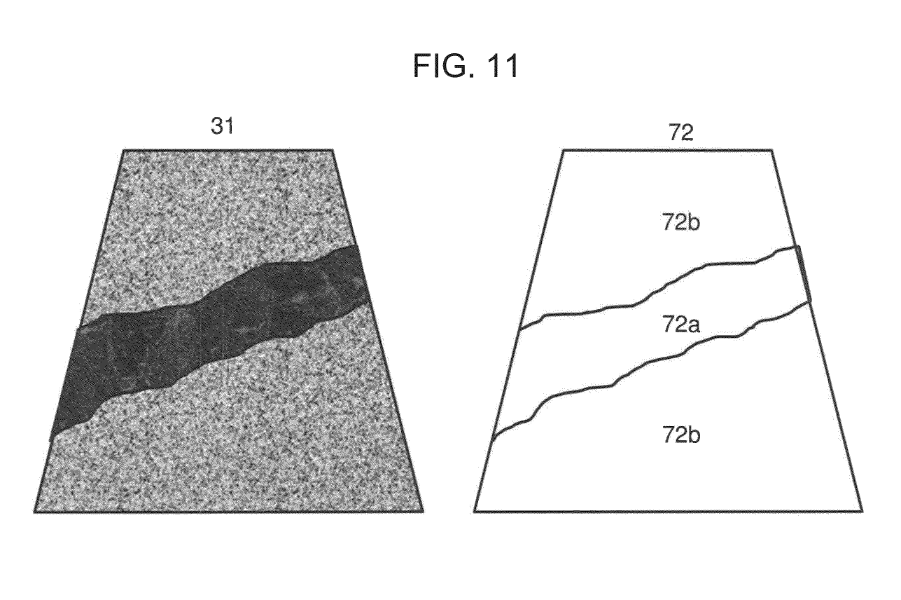

FIG. 11 shows how information from an ultrasonic signal (31), here a power Doppler ultrasound image, according to embodiments, which can then be used in the image reconstruction. The information here is a segmentation of areas where blood is, (72a) and where soft tissues are located (72b). This information can be used as a priori information in the image reconstruction. Details on how to incorporate a priori information in the image reconstruction are, by one of the inventors of this invention are found in the German application 102008025151 of a subgroup of the inventors of this invention and in the German application 102011053708.2.

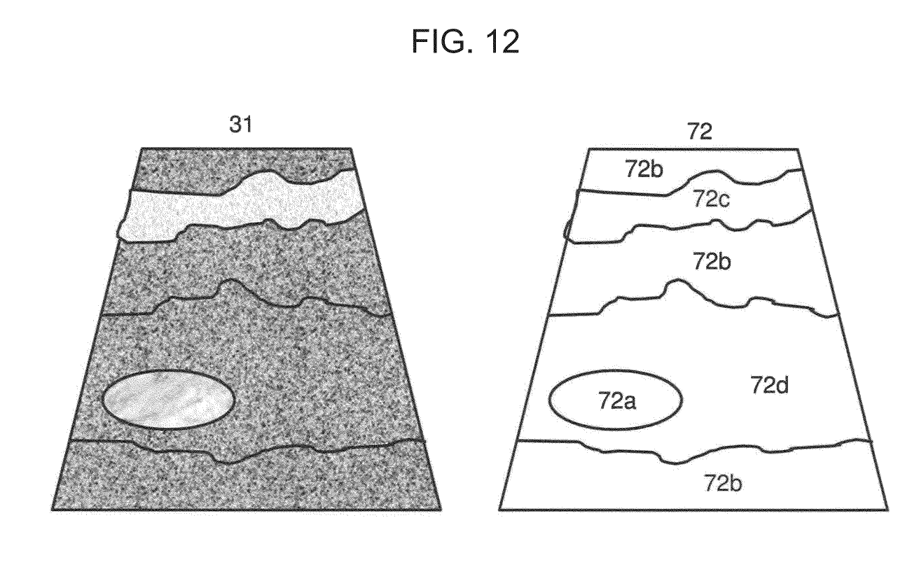

FIG. 12 shows how you can win other information in accordance with embodiments of an ultrasonic signal (31), here a B-mode ultrasound image. In this case, the B-mode ultrasound image is processed and converted by appropriate “look-up tables”, which assign the echogenicity to a X-ray attenuation to a weakening card. The possible processing can be a segmentation of tissues, where a priori knowledge, eg. as the coming into consideration anatomy, ultrasound looks, and what variability of the anatomy (eg in the form, echogenicity, size, etc.) can be expected. The methods to perform this segmentation will not be further specified in this invention, and assumed to be known. From the segmentation result thus different regions (72a, 72b, 72c, 72d) with different attenuations. This attenuation map can then be used in the image reconstruction. Corresponding details on how you can let attenuation maps included in the image reconstruction, have been described in principle for example in German Application 102008025151 and in German application 102011053708.2.

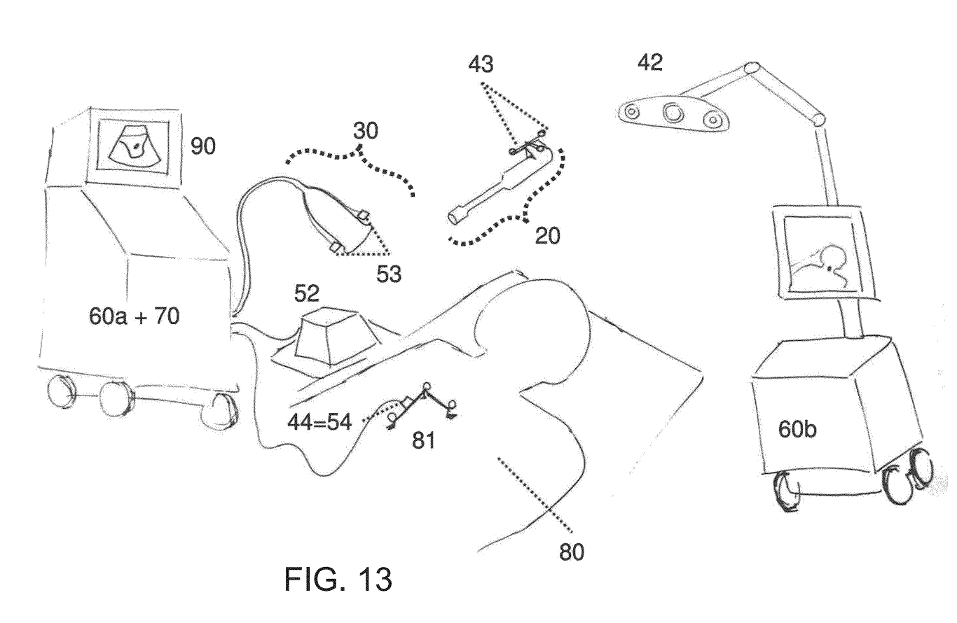

FIG. 13 shows practical implementations of embodiments of the invention. A nuclear detector (20), here a wireless gamma probe sends nuclear detector measurements (21) to the data acquisition module (60) that is subdivided here into two (60a and 60b). passive reflectors on the nuclear Detekor (43) and on the reference from the object or living being (44) the nuclear detector coordinates and reference coordinate. An electromagnetic tracking system, here the ultrasonic tracking system (50), consists of a field generator (52) and electromagnetic sensors on the ultrasound probe (53) and the reference from the object or living being (54). The ultrasonic signals (31), as well as the ultrasonic probe coordinates (51) and the reference coordinates are detected by the data detecting module (60). The complete data is then sent to the reconstruction module (70) and after the image reconstruction on the display (90) to the user.

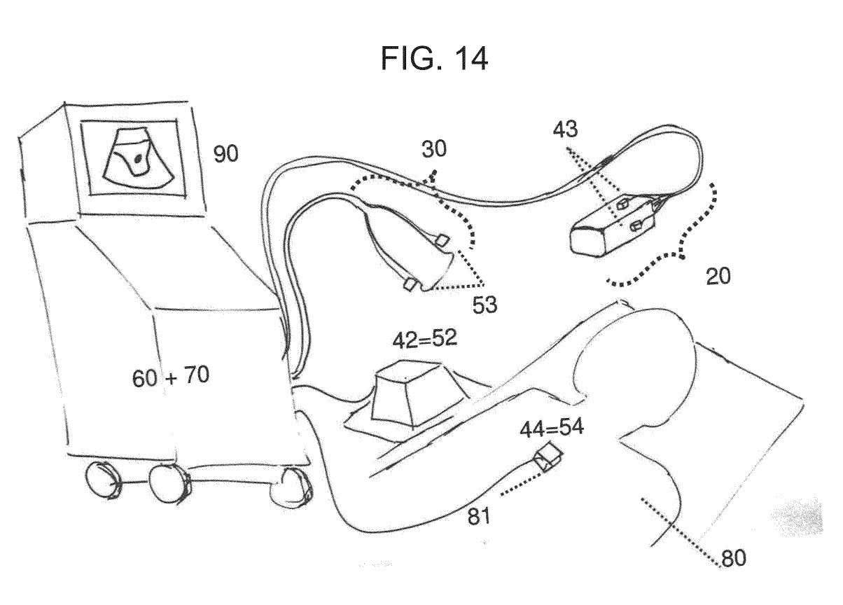

FIG. 14 shows a simplified version of the system of FIG. 13. Here, the nuclear detector (20) comprises a hand-held gamma camera which has no disturbing effects on the electromagnetic tracking system and thus can be tracked by the latter. The nuclear-tracking system (40) and the ultrasonic tracking system (50) in this embodiment, only one.

In embodiments, a combined nuclear and ultrasonic system for hybrid imaging is proposed, which comprises:
a hand-held nuclear radiation detector,
a hand-held ultrasonic probe,
a nuclear tracking system for tracking the nuclear radiation detector while measuring a nuclear radiation and obtaining nuclear radiation detector coordinates representing a position of the tracked nuclear radiation detector in relation to an image coordinate system of a hybrid image,
an ultrasonic tracking system for tracking of the ultrasonic probe during the receiving of ultrasonic signals so that ultrasonic probe coordinates are obtained which represent a position of the tracked ultrasonic probe in relation to an image coordinate system of the hybrid image, and
a data acquisition module, which gathers the nuclear radiation detector measurements, nuclear radiation detector coordinates, ultrasonic signals from the ultrasonic probe, and ultrasonic probe coordinates, and matches them with an image coordinate system of a hybrid image,
a nuclear image reconstruction module for reconstructing a nuclear image from nuclear radiation detector measurements, ultrasonic signals, nuclear radiation detector coordinates and ultrasonic probe coordinates in the image coordinate system of the hybrid image, and
an evaluation system, which calculates a nuclear-image-quality-value of nuclear radiation detector measurements and nuclear radiation detector coordinates.

In further embodiments, the nuclear tracking system and the ultrasonic tracking system are part of the same tracking system.

In further embodiments, the nuclear radiation detector and the ultrasonic probe are integrated in a hand-held probe.

Further embodiments comprise a fixation device for an object or living being.

In further embodiments the nuclear tracking system and the ultrasonic tracking system are each selected from the group consisting of:
Optical passive localization systems,
Optical active localization systems,
Electromagnetic localization systems,
Mechanical localization systems,
Acceleration sensor- or gyroscop q̂
   e-based localization systems,
Sound or ultrasonic localization systems,
Radioactive localization systems,
RF-based tracking systems,
a combination of two or more of the former localization systems.

Further embodiments comprise:
a reference element attached to an object or living being is fixed, and/or attached to another member which is fixed relatively to the object or living being, wherein the reference element is tracked by the nuclear tracking system and by the ultrasonic tracking system.

Further embodiments comprise:
an evaluation system, which calculates a desired position and orientation of the nuclear radiation detector from at least one nuclear-image-quality-value which is calculated continuously from the nuclear radiation detector measurements and nuclear radiation detector coordinates from the previous nuclear radiation, and
a data output system for outputting an instruction to move the nuclear radiation detector to the desired position and orientation to a user and/or a robot.

Further embodiments comprise an interface for receiving:
data pertaining to the surface of the imaged object or living being,
data about a weight distribution of the imaged object or living being, or
a priori image information of the object or living being.

Further embodiments comprise a surgical or interventional instrument which is tracked by the nuclear tracking system, the ultrasonic tracking system, or by both tracking systems.

A method for hybrid imaging is proposed in embodiments, comprising:
detection of nuclear radiation,
tracking of a nuclear radiation detector while measuring a nuclear radiation, such that nuclear radiation detector coordinates are obtained, representing a position of the tracked nuclear radiation detector in relation to an image coordinate system of a hybrid image,
receiving ultrasonic signals,
tracking of an ultrasonic probe during receiving ultrasonic signals, such that ultrasonic signal coordinates are obtained, representing a position of the tracked ultrasonic probe in relation to an image coordinate system of the hybrid image,
acquisition of nuclear radiation detector measurements, nuclear radiation detector coordinates, ultrasonic signals and the ultrasonic probe coordinates, and matching with the image coordinate system of the hybrid image,
reconstructing a nuclear image from nuclear radiation detector measurements, ultrasonic signals, nuclear radiation detector coordinates and ultrasonic probe coordinates in the image coordinate system of the hybrid image, and
calculating a nuclear-image-quality-value from previous nuclear radiation detector measurements and nuclear radiation detector coordinates.

A method for hybrid imaging further comprises calculating the desired position and orientation of the nuclear radiation detector and the subsequent issuing, to a user or a robot, of an instruction to move the nuclear radiation detector to the desired position and orientation.

A method for hybrid imaging further comprises the use of at least one of the following information to reconstruct a nuclear image:
a position of the reference,
a surface of the imaged object or living being,
a weight distribution of the imaged object or living being, or
an a-priori image information of the object or living being.

A method for hybrid imaging further comprises: tracking a surgical or interventional instrument.

A method for hybrid imaging further comprises: displaying the relation between the surgical or interventional instrument and the hybrid image, and/or navigating the surgical or interventional instrument(s) to a position in the hybrid image.

1-10. (canceled)

11. A combined nuclear and ultrasonic system for hybrid imaging, comprising:
a hand-held nuclear radiation detector,
a hand-held ultrasonic probe,
a nuclear tracking system for tracking the nuclear radiation detector while measuring a nuclear radiation and obtaining nuclear radiation detector coordinates representing a position of the tracked nuclear radiation detector in relation to an image coordinate system of a hybrid image,
an ultrasonic tracking system for tracking of the ultrasonic probe during the receiving of ultrasonic signals so that ultrasonic probe coordinates are obtained which represent a position of the tracked ultrasonic probe in relation to an image coordinate system of the hybrid image, and
a data acquisition module, which gathers the nuclear radiation detector measurements, nuclear radiation detector coordinates, ultrasonic signals from the ultrasonic probe, and ultrasonic probe coordinates, and matches them with an image coordinate system of a hybrid image,
a nuclear image reconstruction module for reconstructing a nuclear image from nuclear radiation detector measurements, ultrasonic signals, nuclear radiation detector coordinates and ultrasonic probe coordinates in the image coordinate system of the hybrid image, and
an evaluation system, which calculates a nuclear-image-quality-value of nuclear radiation detector measurements and nuclear radiation detector coordinates.

12. The combined nuclear and ultrasonic system according to claim 11, wherein the nuclear tracking system and the ultrasonic tracking system are part of the same tracking system.

13. The combined nuclear and ultrasonic system according to claim 11, wherein the nuclear radiation detector and the ultrasonic probe are integrated in a hand-held probe.

14. The combined nuclear and ultrasonic system according to claim 11, further comprising a fixation device for an object or living being.

15. The combined nuclear and ultrasonic system according to claim 11, wherein the nuclear tracking system and the ultrasonic tracking system are each selected from the group consisting of:
Optical passive localization systems,
Optical active localization systems,
Electromagnetic localization systems,
Mechanical localization systems,
Acceleration sensor- or gyroscope-based localization systems,
Sound or ultrasonic localization systems,
Radioactive localization systems,
RF-based tracking systems,
a combination of two or more of the former localization systems.

16. The combined nuclear and ultrasonic system according to claim 11, further comprising:
a reference element attached to an object or living being is fixed, and/or attached to another member which is fixed relatively to the object or living being, wherein the reference element is tracked by the nuclear tracking system and by the ultrasonic tracking system.

17. The combined nuclear and ultrasonic system according to claim 11, further comprising:
an evaluation system, which calculates a desired position and orientation of the nuclear radiation detector from at least one nuclear-image-quality-value which is calculated continuously from the nuclear radiation detector measurements and nuclear radiation detector coordinates from the previous nuclear radiation, and
a data output system for outputting an instruction to move the nuclear radiation detector to the desired position and orientation to a user and/or a robot.

18. The combined ultrasonic and nuclear system according to claim 11, further comprising an interface for receiving:
data pertaining to the surface of the imaged object or living being,
data about a weight distribution of the imaged object or living being, or
a priori image information of the object or living being.

19. The combined nuclear and ultrasonic system according to claim 11, further comprising a surgical or interventional instrument which is tracked by the nuclear tracking system, the ultrasonic tracking system, or by both tracking systems.

20. A method for hybrid imaging, comprising:
detection of nuclear radiation,
tracking of a nuclear radiation detector while measuring a nuclear radiation, such that nuclear radiation detector coordinates are obtained, representing a position of the tracked nuclear radiation detector in relation to an image coordinate system of a hybrid image,
receiving ultrasonic signals,
tracking of an ultrasonic probe during receiving ultrasonic signals, such that ultrasonic signal coordinates are obtained, representing a position of the tracked ultrasonic probe in relation to an image coordinate system of the hybrid image,
acquisition of nuclear radiation detector measurements, nuclear radiation detector coordinates, ultrasonic signals and the ultrasonic probe coordinates, and matching with the image coordinate system of the hybrid image,
reconstructing a nuclear image from nuclear radiation detector measurements, ultrasonic signals, nuclear radiation detector coordinates and ultrasonic probe coordinates in the image coordinate system of the hybrid image, and
calculating a nuclear-image-quality-value from previous nuclear radiation detector measurements and nuclear radiation detector coordinates.

21. The method for hybrid imaging according to claim 20, further comprising calculating the desired position and orientation of the nuclear radiation detector and the subsequent issuing, to a user or a robot, of an instruction to move the nuclear radiation detector to the desired position and orientation.

22. The method for hybrid imaging according to claim 20, further comprising the use of at least one of the following information to reconstruct a nuclear image:
a position of the reference,
a surface of the imaged object or living being,
a weight distribution of the imaged object or living being, or
an a-priori image information of the object or living being.

23. The method for hybrid imaging according to claim 20, further comprising: tracking a surgical or interventional instrument.

24. The method for hybrid imaging according to claim 20, further comprising: displaying the relation between the surgical or interventional instrument and the hybrid image, and/or navigating the surgical or interventional instrument(s) to a position in the hybrid image.
